(19) United States
(12) Patent Application Publication
(10) Pub. No.: US 2019/0076472 A1
(43) Pub. Date: Mar. 14, 2019

(54) HYPERAMMONEMIA THERAPY FOR CHILDREN SUFFERING FROM UREA CYCLE DISORDERS

(71) Applicant: CT Development One, LLC, Cincinnati, OH (US)

(72) Inventor: Ronald J. THOMPSON, Cincinnati, OH (US)

(21) Appl. No.: 15/704,174

(22) Filed: Sep. 14, 2017

(51) Int. Cl. A61K 33/44; A61K 9/50; B01J 20/20; B01J 20/28; A61K 9/00; A61K 31/7016

(52) U.S. Cl. CPC A61K 33/44; A61K 9/5073; A61K 31/7016; B01J 20/28016; A61K 9/0053; B01J 20/20

(57) ABSTRACT

A method and composition for treating or preventing the progression of hyperammonemia caused by a Urea Cycle Disorder, the method comprising administration of an effective amount of porous activated carbon particles, wherein the porous activated carbon particles are enteric-coated in order to control their release and adsorption properties. The porous activated carbon particles or microspheres can initially be coated with lactulose, followed by enterically coating the lactulose-covered carbon particles. The inventive method and composition provides a safe and uncomplicated reduction of ammonia levels in affected children.

### FIELD OF THE INVENTION

This invention relates in general to the treatment of hyperammonemia, and in particular to therapy for reducing toxic ammonia levels in newborns, infants and children suffering from a Urea Cycle Disorder.

### BACKGROUND OF THE INVENTION

Ammonia is generated during metabolism of proteins in all mammalian organs, and, without a means to metabolize it, can be highly toxic. The healthy liver actively processes ammonia via the urea cycle and thus prevents excess amounts of ammonia from entering the systemic circulation. Ammonia is converted to non-toxic molecules that are excreted in the urine by the kidneys. However, if some part of the ammonia detoxifying mechanism is abnormal, large amounts of ammonia accumulate in the upper digestive tract, and blood ammonia levels increase. As protein uptake continues, manifestations of hyperammonemia begin to occur.

Hyperammonemia can be extremely damaging to the brain. Acute onset of hyperammonemia, especially in newborns, infants and children, can cause acute neurological manifestations such as seizures, ataxia, vision loss, coma, psychosis, acute encephalopathy, cerebral edema, respiratory alkalosis, hypothermia, brain damage, and (if left untreated) death. Chronically elevated ammonia levels can cause mood disturbances, insomnia, fatigue, loss of coordination/dexterity, clumsiness, confusion and inability to concentrate, as well as nausea, vomiting, headaches, diarrhea, back pain and accelerated aging.

Unfortunately, some individuals have particular genetic mutations which do not allow them to effectively metabolize and eliminate ammonia. One area of concern represents a substantial risk of brain damage and death among newborns and infants. In neonatal-onset Urea Cycle Disorders (UCDs), which are characterized as inborn errors of metabolism, one of six specific urea cycle enzymes (i.e. arginase, carbamoylphosphate synthetase, N-acetylglutamate synthetase, omithine transcarbamylase, Argininosuccinic Acid Synthetase and Argininosuccinate Lyase) are defective, deficient, or completely absent. All of the urea cycle enzymes, except ornithine transcarbamylase, are transmitted genetically as autosomal recessive genes, i.e. each parent contributes a gene to the child. Omithine transcarbamylase deficiency is a rare X-linked genetic disorder.

The estimated incidence of UCD is 1 in every 8500 births, but many cases remain undiagnosed and/or infants born with the disorders die without a definitive diagnosis. About half of the diagnoses are made in the first week of life, and about 90% of all UCDs are diagnosed by age 7, with the remainder by 18 years of age. Patients diagnosed after the first week of life typically have only a partial genetic defect in the urea cycle. Older individuals are much harder to diagnose, but still are at risk of the long term mental consequences of hyperammonemia and its neurotoxicity. In all, there are approximately 60 UCD diagnoses per 1 million US children/year, 240 US children with UCD diagnosed/year, and about 1680 diagnosed in past 7 years. The current number of US children diagnosed with UCD is estimated to be between 1500 to 1800. Further, it is believed that up to 20% of Sudden Infant Death Syndrome (SIDS) cases may be attributed to an undiagnosed inborn error of metabolism such a UCD, and some children with autism spectrum disorder or other behavioral disorders may have undiagnosed UCDs. Thus, the exact incidence of neonatal UCDs is unknown and underestimated.

Although there is no cure, liver transplant permanently corrects these disorders in most cases, but is typically used only as a last resort where non-surgical treatment has proven insufficient. In newborns suffering from an inherited UCD, about half present with hyperammonemia in the first week of life. The diagnosis constitutes a medical emergency because the rising levels of ammonia in the newborn's bloodstream will quickly lead to neurotoxicity, encephalopathy, and a risk of lifelong mental retardation, if not neonatal death. Prompt hemodialysis is often needed to immediately reduce the hyperammonemia.

Current therapies for hyperammonemia and UCDs aim to reduce ammonia excess, but are widely regarded as suboptimal. Most UCD patients require supplementation with ammonia scavenging drugs, such as oral sodium phenylbutyrate (Buphenyl®) and glycerol phenylbutyrate (Ravicti®), or injectable sodium phenylacetate in combination with sodium benzoate (Ammonul®). Typically one or more of these drugs must be administered three to four times per day, and include side effects such as nausea, vomiting, irritability, and anorexia. In addition, many patients require substantially modified diets consisting of protein restriction, these drugs are typically not useful for treatment of acute hyperammonemia, and they are not indicated for patients less than two months of age. When these treatment options fail, a liver transplant may be the only option.

In light of the above, it is apparent that there is a limited number of treatment options available for patients, and especially for newborns, infants and children, suffering from hyperammonemia resulting from a Urea Cycle Disorder (UCD). Thus a significant unmet need exists for an effective, reliable and long-term therapy for hyperammonemia in UCD patients.

### SUMMARY OF THE INVENTION

The present invention is an improved composition and method which employs enteric-coated activated carbon for both acute therapy and progression prevention therapy of hyperammonemia in children suffering from a Urea Cycle Disorders (UCDs).

A first aspect of the invention relates to a method of treating or preventing the progression of hyperammonemia caused by a Urea Cycle Disorder in a child, the method comprising: orally administering an effective amount of porous activated carbon particles, wherein the particles are enteric-coated in order to control their release and adsorption properties.

A second aspect of the invention relates to a method of treating or preventing the progression of hyperammonemia caused by a Urea Cycle Disorder in a child, the method comprising: orally administering an effective amount of porous activated carbon particles, wherein the particles are enteric-coated in order to control their release and adsorption properties, in combination with oral administration of sodium phenylbutyrate (Buphenyl®) or glycerol phenylbutyrate (Ravicti®), or injectable administration of sodium phenylacetate and sodium benzoate (Ammonul®), as a dual therapy.

A third aspect of the invention relates to an orally administered composition for use in treating or preventing the progression of hyperammonemia caused by a Urea Cycle Disorder, the composition comprising porous activated carbon particles, wherein the porous activated carbon particles are enteric-coated.

While the nature and advantages of the present invention will be more fully appreciated from the following drawings and detailed description, showing the contemplated novel construction, combinations and elements as herein described, and more particularly defined by the appended claims, it is understood that changes in the precise embodiments of the present invention are meant to be included within the scope of the claims, except insofar as they may be precluded by the prior art.

### BRIEF DESCRIPTION OF THE DRAWINGS

FIG. 1 is a perspective view of the stomach and small intestines of a newborn being dosed with an activated carbon suspension according to the invention;

FIG. 2 shows the stomach and intestines of FIG. 1 approximately 1 hour after dosing;

FIG. 3 shows the stomach and intestines of FIG. 1 approximately 3 hours after dosing.

### DETAILED DESCRIPTION OF THE INVENTION

As used herein, the terms “child”, “children” and “pediatric” are considered to refer to and include newborns, neonates, infants, toddlers, and children up to 7 years of age. A newborn, or neonate, is a baby under 28 days old. Infants are typically about 1 month to about 9-12 months old, toddlers are typically about 9-12 months to about 2 years of age. The present invention is intended for use with newborns, neonates, infants, toddlers, and children up to 7 years old suffering from a urea cycle disorder.

The terms “activated carbon” or “activated charcoal” as used herein refer to particles or granules of highly porous charcoal product that has been “activated” with steam or acid. The activation process carves away the internal structure of the charcoal particles, producing pores having a high (internal) surface area which attracts and holds organic chemicals inside it. For the purposes of the present invention the porous activated carbon is preferably made from coconut shell, but it can also be made from different sources such as wood, bamboo, or coal.

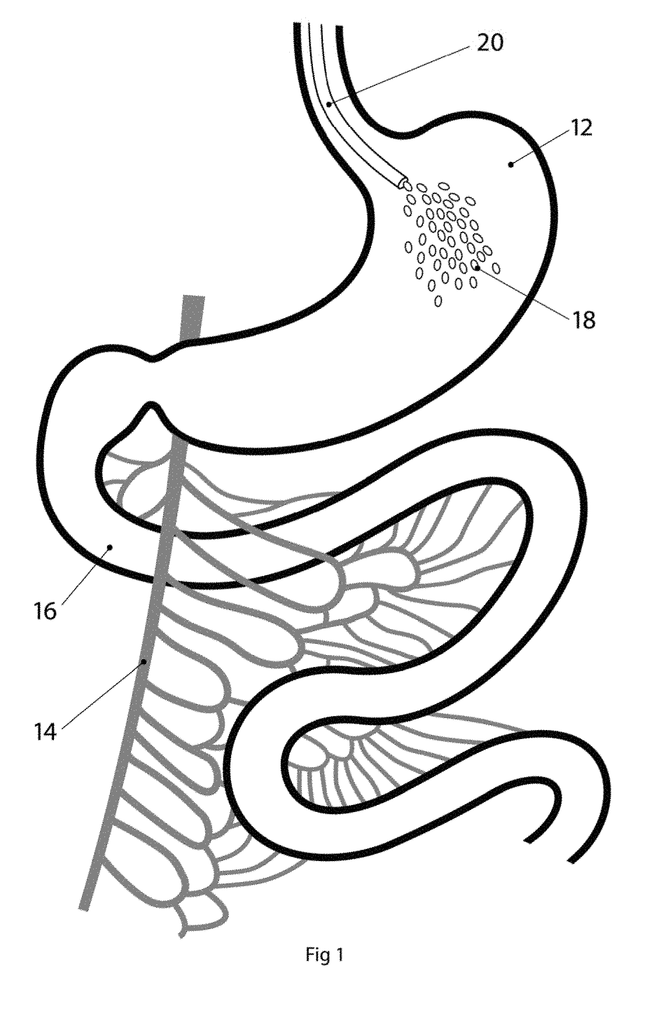

Referring now to the drawings in detail, FIG. 1 shows the stomach 12 and surrounding vasculature 14 and small intestines 16 of a child. The small intestine 16 (i.e. small bowel, upper intestines) is a tube that lies between the stomach 12 and the large intestine (i.e. large bowel, lower intestines, not shown) and includes the duodenum, jejunum, and ileum. The small intestine is so called because its lumen is smaller (about 2.5 cm in diameter) than that of the large intestine, although it is actually longer in length than the large intestine. The small intestine 16 is fed by vasculature 14 including the inferior mesenteric vein and extends from the pyloric sphincter of the stomach to the ileocecal valve, where it joins the large intestine. The adult ileum is about 350 cm long, with the adult duodenum/jejunum combined being ˜250 cm long, so that the entire the entire adult small intestine is approximately 600 cm (20 feet) long and coiled in loops filling most of the abdominal cavity. In contrast, the newborn ileum is only 250 cm long, with the duodenum/jejunum combined being ˜50 cm long, so that the entire newborn small intestine is only approximately 300 cm (10 feet) long.

The use of activated charcoal for medicinal purposes dates back centuries. Indeed, non-enterically coated activated carbon is a common and effective detoxification agent, used daily in hospitals for treating accidental poisoning and overdose emergencies. Activated charcoal acts as a chemical sponge, absorbing toxic chemicals before they can be absorbed into the bloodstream by the stomach. A suspension of activated charcoal (non-enterically coated) in combination with magnesium (i.e. a charcoal-magnesium flush) has been used to reduce hyperammonemia in patients with cystathionine beta synthase (CBS) enzyme deficiency. See Dr. Amy Yasko, “Autism: Pathways to Recovery” Copyright 2004, Neurological Research Institute, ISBN: 978-1-4243-4320-1.

While non-enterically coated activated carbon is useful as described above, the pores of the activated carbon particles are immediately exposed to chemicals within the stomach 12. Such immediate, non-specific absorption of chemical compounds in the stomach by the activated carbon is ineffective for the purposes of the present invention. That is, if there is no enteric coating then the surface area of the activated carbon immediately begins to indiscriminately absorb albumin, fats and other organic and toxic chemical compounds in the stomach. Since the high levels of ammonia are not encountered until the small intestine, the inventor has found the use of non-enterically coated activated carbon for scavenging ammonia to be inefficient. The activated carbon is more effective if its release and adsorption properties can be controlled. Thus, the activated carbon used in the present invention is enteric-coated and intended to be released downstream from the stomach 12, in the small intestines 16, where the large surface area provided by the porous activated carbon particles 18 can efficiently chelate and/or absorb the targeted ammonia toxins 24.

Most enteric coatings work by presenting a surface, such as a press-coated cellulose polymer layer as is known in the art (and discussed in more detail below). This layer is stable at the acidic, low pH found in the stomach, but breaks down rapidly at a higher pH. The intraluminal pH of the intestinal tract is essentially the same in children and adults, rapidly changing from a highly acidic pH in the stomach (between pH 1.5 and 3.5) to about pH 6 in the duodenum. The pH gradually increases in the small intestine from pH 6 to about pH 7.4 in the terminal ileum. The acid resistance of the enteric coating prevents the porous activated carbon particles from being released in the stomach. The enteric coating allows predominant release of active carbon particles into the small intestines, where the carbon particles are more useful for the intended purpose of scavenging ammonia.

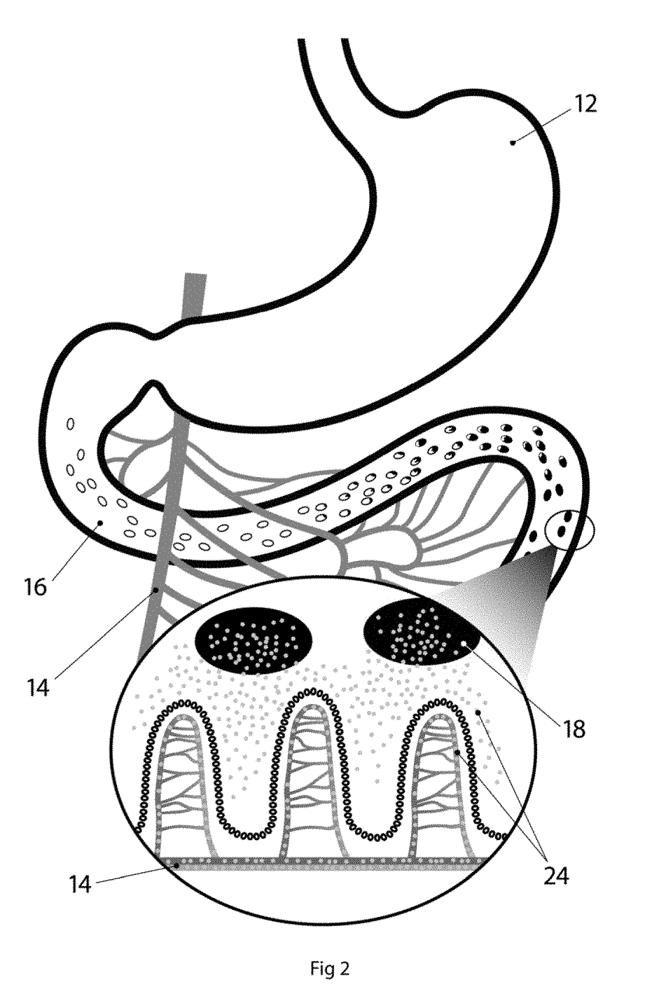

FIG. 2 illustrates the child's stomach 12 (now empty of enteric-coated porous activated carbon particles 18) and small intestines 16 approximately one hour after dosing. Following gastric emptying, the enteric coating is dissolved in the higher, more alkaline pH of the small intestine 16, and the newly uncoated porous activated carbon particles (shown in black, as opposed to the enterically-coated carbon particles shown in white) are now free to chelate and/or absorb the high levels of ammonia 24 in the small intestine. The inset in FIG. 2 shows a close-up view of the junction between the mesenteric vasculature 14 and the inside lumen of the small intestine 16, where high levels of ammonia 24 resulting from the child's urea cycle disorder are being deposited in the intestinal chyme. Ammonia (NH₃) is dissolved in the blood plasma, and the concentration of ammonia equilibrates by simple diffusion between the blood plasma in the vasculature 14 and the lumen of the small intestine 22.

Generally, for adults, blood ammonia levels between 9 and 50 micrograms per deciliter (mcg/dL) is considered normal. The levels of ammonia considered normal in children are between 40 and 80 mcg/dL. In newborns, the levels of ammonia should be between 90 and 150 mcg/dl. In children (i.e. newborns, infants and children up to age 7 years) suffering from UCD, the concentration of ammonia is dangerously high and can reach between 150-350 mcg/DL. The porous activated carbon particles act to absorb, chelate and/or entrap the ammonia within the lumen of the small intestine, preventing the ammonia from entering the bloodstream and thus acting as an ammonia sink. As the ammonia 24 is absorbed into the pores of the activated carbon 18, the concentration of ammonia drops in the lumen of the small intestine, causing an equivalent concentration drop of ammonia in the blood plasma.

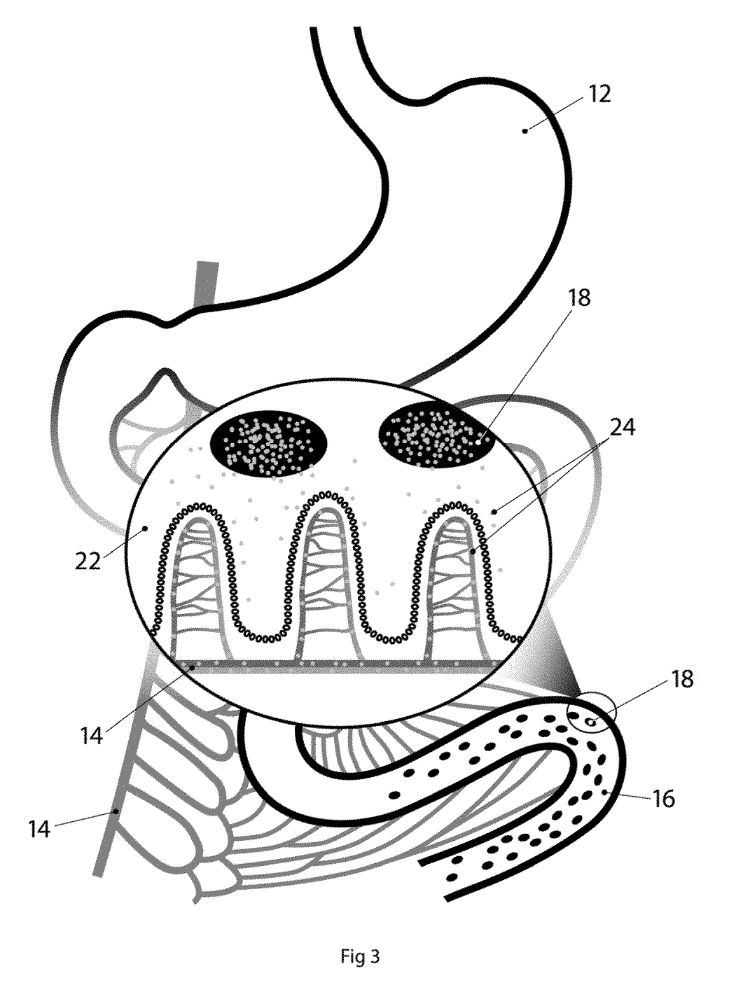

FIG. 3 illustrates the child's stomach 12 and small intestines 16 approximately three hours after dosing. The uncoated activated carbon particles 18 have now reached the distal ileum of the small intestines 16, and they are loaded with ammonia molecules 24. The concentration of ammonia in both the intestinal lumen 22 and the blood plasma of the mesenteric vasculature 14 is significantly reduced. From here, the ammonia-laden carbon particles 18 are passed through the large intestine (not shown) and eliminated in the feces via the rectum during a bowel movement. The activated carbon particles 18 are not absorbed systemically, instead simply passing through the intestines 16 while picking up large amounts of ammonia 24 along the way and detoxifying the patient. The enteric coating allows the activated carbon particles 18 to begin working after gastric emptying, so that the particles preferentially absorb ammonia residing in the small intestine.

The present invention envisions using a suspension of spherical, porous activated carbon particles, or microspheres of activated carbon. As a non-limiting example, the activated carbon for use with the inventive method can be dry packaged in packets containing the enteric-coated porous activated carbon, for preparation as a suspension prior to administration to the patient. As another non-limiting example, the activated carbon can be prepared as a suspension containing between 0.1 gram and 4 grams of enteric-coated porous activated carbon particles per deciliter of suspension. Further, the activated carbon can be prepared as a suspension containing about 1 gram of enteric-coated porous activated carbon particles per deciliter of suspension.

More specifically, the inventive method can use USP Grade 4 microporous activated carbon compressed into microspheres 30-50 microns in external diameter, and having an enteric coating between 2 and 20 microns in thickness. The location in the small intestine where the enteric coating is dissolved from the activated charcoal may be controlled by either the thickness of the particular enteric coating used, or by the type of enteric coating used. Such manufacturing techniques are well known to those skilled in the art.

As a non-limiting example, the spherical activated carbon particles can be covered with a thin enteric coating approximately 2-4 microns thick, such that the activated charcoal is predominantly released in the proximal small intestine (the duodenum, jejunum, and proximal ileum). In another example, the spherical activated carbon particles can be covered with a thick enteric coating approximately 8-9 microns thick, such that the activated charcoal is predominantly released in the distal or lower third of the small intestine. In another non-limiting example, the spherical activated carbon particles can be covered with one of two different thicknesses of enteric coating; for example, the first coating can be a thin coating approximately 2-4 microns thick, and the second coating can be a thick coating approximately 8-9 microns thick, with approximately half of the activated carbon particles having the thin coating and the remainder having the thick coating.

As another non-limiting example, the carbon particles can be covered with two different types of enteric coating, with approximately half of the activated carbon particles having an enteric coating that predominantly dissolves in the proximal small intestine and the remainder having a different type of enteric coating that predominantly dissolves in the distal or lower third of the small intestine. These means of enterically coating the activated carbon (i.e. differentially changing either the thickness or the type of enteric coating used) can ensure that the first enteric coating will dissolve and expose the activated carbon particles in the duodenum, jejunum, and proximal ileum, while the second enteric coating will dissolve and expose the remaining activated carbon particles in the remainder of the ileum, providing a prolonged exposure to activated carbon particles along the entire length of the small intestine.

The inventive method thus provides maximum exposure of the activated carbon to the excess ammonia in the small intestine while limiting the ability of the activated charcoal from absorbing and becoming overloaded with other organic chemicals and toxins in the stomach prior to performing its intended pharmaceutical function. Non-limiting examples of different types of polymer film layers/coatings which may be used for enterically coating the porous activated charcoal include hydroxypropyl cellulose (HPC), hydroxypropyl methylcellulose (HPMC), cellulose acetate phthalate, methylacrylic acid co-polymer type C, and methylacrylic acid co-polymer type A. Many enteric coatings are known for both soft gel capsules and hard capsules and can be useful for manufacturing the enteric coatings of the present invention, as disclosed for example in U.S. Pat. No. 9,254,270 to Hassan et al., U.S. Pat. No. 9,241,911 to Miller, U.S. Pat. No. 9,198,868 to Benameur et al., U.S. Pat. No. 5,672,359 to Digenis et al., U.S. Pat. No. 5,330,759 to Pagay et al., and U.S. Pat. No. 4,138,013 to Okajima, all of which are incorporated herein by reference in their entirety.

In one embodiment, the porous activated carbon particles or microspheres can initially be coated with lactulose, followed by enterically coating the lactulose-covered carbon particles. The inventor has found that the standard approach of enterically coating granules or particles of activated charcoal can decrease the efficacy the activated charcoal, especially when highly porous activated charcoal is used. Specifically, if small amounts of porous activated charcoal are shaped into granules and then enterically coated, upon ingestion the enteric coating does not fully dissolve out of the pores, thereby reducing the available surface area of the activated charcoal and decreasing or preventing its effectiveness in chelating ammonia. Therefore, a novel method for preparing the activated charcoal for the present invention includes first coating the small porous activated charcoal granules with lactulose. Lactulose is currently used for treating hyperammonemia, and works by transforming ammonia into ammonium ion (NH⁺⁴) which can no longer diffuse back into the blood. Lactulose coating of the activated carbon particles prior to enterically coating the particles can prevent the enteric coating from filling/clogging the pores of the activated carbon particles while also decreasing the amount of ammonia. Further, lactulose can ameliorate constipation, which often results from administration of an activated carbon suspension.

In another embodiment, menthol can be packaged as a microbial preservative agent along with the activated carbon particles. In another embodiment, a buffer or acid, such as citric acid, can be used to maintain the suspension of activated carbon at a pH of about 5, so that the enteric coating is not dissolved prior to administration. In use, a dedicated hospital pharmacist or other qualified individual can prepare a suspension for administration to the patient. In the case of a newborn/infant, the suspension can be delivered by lavage or feeding tube, and with older children a baby bottle may be used. Upon patient discharge, the pharmacist can instruct the parents or home health care personnel regarding suspension preparation for home therapy.

The enterically coated porous activated carbon used in the inventive method is advantageous over the use of medications such as oral sodium phenylbutyrate (Buphenyl®) and glycerol phenylbutyrate (Ravicti®), or injectable sodium phenylacetate in combination with sodium benzoate (Ammonul®) in UCD Patients, because it is not absorbed into the blood (i.e. non-systemic), it can be used in newborns and infants less than 2 months of age (medications such as sodium phenylbutyrate (Buphenyl®) and glycerol phenylbutyrate (Ravicti®) are contraindicated in patients less than 2 months of age), it is eliminated in the bowel (i.e. not systemically absorbed and not renally excreted), and can be used in UCD patients with pancreatic insufficiency and/or intestinal malabsorption issues. The composition of lactulose and porous activated carbon particles disclosed herein can also be used for treatment of acute hyperammonemia, unlike other oral medications for treating UCD. The inventive method and composition also prevents the risk of phenylacetate neurotoxicity (PAA, a metabolite of phenylbutyrate), seen with other medications such as sodium phenylbutyrate (Buphenyl®) and glycerol phenylbutyrate (Ravicti®), and also has no risk of hypernatremia, hypokalemia, or water intoxication. In one embodiment of the invention, the enteric-coated porous activated carbon used in the method described herein can be combined with traditional therapies for hyperammonemia, including oral administration of lactulose, sodium phenylbutyrate (Buphenyl®) or glycerol phenylbutyrate (Ravicti®), or injectable administration of sodium phenylacetate in combination with sodium benzoate (Ammonul®). This dual therapy can enhance the efficiency of ammonia reduction while decreasing the side effects of the traditional therapies.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative system and method, and illustrated examples shown and described. Accordingly, departures may be made from such details without departing from the scope of the invention.

What is claimed is:

1. A method of treating or preventing the progression of hyperammonemia caused by a Urea Cycle Disorder in a child, the method comprising: orally administering an effective amount of porous activated carbon particles, wherein the particles are enteric-coated in order to control their release and adsorption properties.

2. The method of claim 1, wherein the child is less than 7 years old.

3. The method of claim 1, wherein the child is less than 2 years old.

4. The method of claim 1, wherein the child is less than 12 months old.

5. The method of claim 1, wherein the child is less than 2 months old.

6. The method of claim 1, wherein the child is less than 28 days old.

7. The method of claim 1, wherein the enteric-coated porous activated carbon particles are administered as a suspension of porous activated carbon particles.

8. The method of claim 1, wherein approximately half of the carbon particles have a first enteric coating which dissolves and exposes the activated carbon particles in the duodenum, jejunum, and proximal ileum, and wherein approximately half of the carbon particles have a second enteric coating which dissolves and exposes the remaining activated carbon particles in the remainder of the ileum, providing a prolonged exposure to activated carbon particles along the entire length of the small intestine.

9. The method of claim 8, wherein the first enteric coating is a thin coating, and wherein the second enteric coating is a thick coating.

10. The method of claim 8, wherein the first enteric coating is a first type of enteric coating and the second enteric coating is a second type of enteric coating.

11. The method of claim 1, wherein the enteric-coated porous activated carbon is dry packaged in packets for preparation as a suspension prior to administration to the child.

12. The method of claim 5, wherein menthol is packaged with the enteric-coated porous activated carbon particles as a microbial preservative agent.

13. The method of claim 1, wherein the step of administering an effective amount of porous activated carbon particles is performed in combination with oral administration of sodium phenylbutyrate (Buphenyl®) or glycerol phenylbutyrate (Ravicti®), or injectable administration of sodium phenylacetate and sodium benzoate (Ammonul®), as a dual therapy.

14. A method of treating or preventing the progression of hyperammonemia caused by a Urea Cycle Disorder in a child, the method comprising: orally administering an effective amount of porous activated carbon particles, wherein the particles are enteric-coated in order to control their release and adsorption properties, in combination with oral administration of sodium phenylbutyrate (Buphenyl®) or glycerol phenylbutyrate (Ravicti®), or injectable administration of sodium phenylacetate and sodium benzoate (Ammonul®), as a dual therapy.

15. An orally administered composition for use in treating or preventing the progression of hyperammonemia caused by a Urea Cycle Disorder, the composition comprising porous activated carbon particles, wherein the porous activated carbon particles are enteric-coated.

16. The composition of claim 15, further comprising lactulose, wherein the porous activated carbon particles are initially coated with lactulose, followed by enterically coating the lactulose-covered porous activated carbon particles.

17. The composition of claim 15, wherein the composition is administered as a suspension of porous activated carbon particles.

18. The composition of claim 15, wherein approximately half of the carbon particles have a first enteric coating which dissolves and exposes the activated carbon particles in the duodenum, jejunum, and proximal ileum, and wherein approximately half of the carbon particles have a second enteric coating which dissolves and exposes the remaining activated carbon particles in the remainder of the ileum, providing a prolonged exposure to activated carbon particles along the entire length of the small intestine.

19. The composition of claim 18, wherein the first enteric coating is a thin coating, and wherein the second enteric coating is a thick coating.

20. The method of claim 18, wherein the first enteric coating is a first type of enteric coating and the second enteric coating is a second type of enteric coating.
